(19) United States
(12) Patent Application Publication
(10) Pub. No.: US 2024/0382342 A1
(43) Pub. Date: Nov. 21, 2024

(54) LIQUID DISPENSER

(71) Applicant: Aptar Radolfzell GmbH, Radolfzell (DE)

(72) Inventor: Jürgen GREINER-PERTH, Gottmadingen (DE)

(21) Appl. No.: 18/689,901

(22) PCT Filed: Sep. 9, 2022
(86) PCT No.: PCT/EP2022/075093

§ 371 Date: Mar. 7, 2024

(30) Foreign Application Priority Data
Sep. 10, 2021 (EP) 21196124.8

(51) Int. Cl. A61F 9/00

(52) U.S. Cl. CPC A61F 9/0008

(57) ABSTRACT

A drop dispenser for discharging a liquid in drops. The drop dispenser has a liquid reservoir for storing the liquid prior to discharge and a discharge opening associated with a drop-forming geometry. The drop dispenser has a pump device for pumping liquid out of the liquid reservoir and to the discharge opening so that the liquid collects on the drop-forming geometry and is released from the drop-forming geometry when a sufficient quantity of liquid is collected. The drop dispenser has a flat housing, within which the liquid reservoir and the pump device are arranged, and the outer shape of the flat housing has a maximum width orthogonally to a main axis of the housing, the maximum width being at least 25% greater than a maximum thickness of the outer shape of the housing orthogonally to the main axis and orthogonally to the width. An actuation handle actuates the pump device.

### FIELD OF APPLICATION AND PRIOR ART

The invention relates to a drop dispenser for discharging a liquid in drops, in particular for discharging a pharmaceutical liquid in drops. Such a drop dispenser can be used, for example, for ophthalmic purposes.

A drop dispenser according to the invention, like drop dispensers of the type in question, has a liquid reservoir for storing the liquid prior to the discharge process and a pump device, by means of which liquid can be pumped out of the liquid reservoir and to a discharge opening of the drop dispenser. The discharge opening is associated with a drop-forming geometry so that the liquid can collect on this drop-forming geometry and can be released from the drop-forming geometry as individual drops when a sufficient quantity of liquid is collected.

Such drop dispensers permit a discharge of liquid which can be accurately metered.

### OBJECT AND SOLUTION

It is the object of the invention to provide a drop dispenser which can be carried easily and safely in everyday life, and which is easy to use.

According to the invention, a drop dispenser of the type in question is proposed to this end, the drop dispenser having the described components of a liquid reservoir, a pump device and a discharge opening. When ready for sale, the liquid reservoir is preferably filled with a pharmaceutical liquid. In particular, the liquid can be a liquid which is free of preservatives, wherein the dispenser is designed for use with such a liquid which is free of preservatives, such that an ingress of germs from the outside is effectively prevented, in particular by a securely closing discharge valve and/or by a ventilation channel which is provided with a sterile filter, by means of which at least 99.9% of the germs are removed from the incoming air. The ventilation channel can also be provided with a ventilation valve.

The discharge opening is provided in the region of a drop-forming geometry which is suitable for forming individual drops of a size which is substantially reproducible. For example, it can be a cup-shaped drop-forming surface or a drop-forming surface which is surrounded by a separation edge and which is substantially planar. In the non-use state, the discharge opening and the drop-forming geometry are preferably protected by a protective cap which can be pushed onto the housing.

A particularly compact design in which the drop dispenser has a flat housing is primarily proposed. This flat housing contains the liquid reservoir and at least the essential part of the pump device. All of the structurally significant parts of the dispenser which remain stationary relative to one another during operation are regarded as the housing within the meaning of the invention.

A flat design is understood to mean a design in which a maximum width of the housing exceeds the maximum thickness of the housing by at least 25%, preferably by at least 50%, in particular preferably by 100% or more. In particular, it is preferred if the housing is twice as wide as it is thick. The length of the housing preferably exceeds the width, in particular preferably by at least 50%.

Such a flat housing can be carried comfortably and, in particular, is suitable for being stowed in a trouser pocket. Preferably, it is a relatively small dispenser with a width of preferably 3.6 cm or more, but in particular preferably less than 6 cm. The thickness of the housing is preferably less than 1.8 cm, in particular preferably 1.5 cm or less. The length of the housing is preferably at least 4 cm, but in particular is preferably 5 cm or more. The length preferably does not exceed 10 cm, and in particular is preferably 8 cm or less.

The main axis of a drop dispenser according to the invention extends from a reservoir bottom to the discharge opening and/or is defined by the orientation of the discharge opening or a central axis of the liquid reservoir.

Preferably, relative to the viewing direction of the main axis, a cross section of the housing is provided with a substantially elliptical cross section, at least in a partial region of preferably at least 40% of the length of the housing. This is understood to mean that the cross section is not designed to be rectangular or rectangular with corners rounded in a shape of a quarter circle, but has a non-uniform radius of curvature which corresponds to an ellipse and which is larger in the region of a front face and a rear face relative to the short lateral surfaces.

This elliptical shape is particularly advantageous in a compact dispenser of the type according to the invention since the rear face can bear with a correspondingly gently rounded shape securely against the index finger, the middle finger and the ring finger, when an actuating surface arranged opposingly on the front face is pressed in. This promotes the ability to position the drop dispenser accurately at the same time and then to be able to actuate it reliably and sensitively.

The compact outer dimensions of the housing, however, are preferably combined with a still relatively large liquid reservoir. The internal volume of the liquid reservoir is preferably at least 5 ml, in particular preferably at least 10 ml. The internal volume of the liquid reservoir is preferably no more than 30 ml.

An internal volume of the liquid reservoir preferably is at least 50%, preferably at least 60%, of the volume enclosed by an outer contour of the drop dispenser, possibly with the protective cap removed. Preferably, at least half of the volume of the drop dispenser is formed by the internal volume of the liquid reservoir. The volume enclosed by the outer contour of the drop dispenser without the cap is preferably between 10 cm³ and 60 cm³, in particular between 15 cm³ and 30 cm³.

This aspect of the invention relating to the ratio between the internal volume and the external volume can also be advantageous without the described flat design, wherein the further sub-aspects described in this description of the dispenser can also be provided in such a dispenser without a flat design.

In order to achieve a large volume of the liquid reservoir in relation to the volume of the outer contour, it can be advantageous if the liquid reservoir extends in the direction of the main axis into the region of the pump device, so that the pump device, in particular the pump chamber thereof, is located at the same level as the liquid reservoir relative to the main axis. In particular, relative to the width of the housing, the liquid reservoir can have in each case a partial portion of the internal volume of the liquid reservoir on the left-hand side and on the right-hand side of the pump chamber. In addition to the fact that an overlapping design makes ideal use of the available installation space, a very wide inlet into the pump chamber can also be achieved in a very simple manner by the pump chamber overlapping with the liquid reservoir, and this helps to prevent air from collecting therein which could disrupt the suctioning of liquid by the pump device. The inlet between the liquid reservoir and the pump chamber is preferably not less than 2 mm² at the narrowest point.

In a drop dispenser according to the invention, the discharge takes place by means of the aforementioned pump device. This pump device is provided inside the housing and is actuated by means of an actuation handle which can be pressed in and preferably is pivotably movable. If the drop dispenser has a protective cap, this is preferably adapted such that in the positioned state it covers the actuation handle or otherwise prevents an undesired actuation of the actuation handle.

The actuation handle can be provided on a lateral surface which spans the thickness of the dispenser. This design can be advantageous relative to the design of the pump device, since the pump device can then be designed very simply with a piston which is movable in the transverse direction, wherein a relatively large piston stroke is possible.

In order to promote a compact design of the dispenser and a comfortable actuation, however, it is preferred if the actuation handle is provided on a front face of the housing which spans the width, in particular in a centered arrangement. An actuation handle which is provided here can be relatively large in spite of the flat design of the dispenser and thus can permit a pleasant and accurately metered actuation. The actuation handle is preferably arranged on the front face in a centered arrangement relative to the width. The actuation handle preferably takes up more than half of the available space on the front face relative to the width. In particular, the width of the actuation handle is preferably at least 20 mm, in particular at least 25 mm. In particular, with such a relatively large actuation handle arranged on the front face it is advantageous if the protective cap can cover the actuation handle in a protective manner and is preferably also sufficiently rigid that an undesired actuation of the actuation handle is prevented when the protective cap is in position.

The actuation handle preferably acts on a pump device with a piston unit which is movable orthogonally to the main axis and to the width. This is because due to the thin design the space for such a pump device, and in particular the pump stroke thereof, is very limited here. The small discharge quantity per actuation of preferably between 5 μl and 30 μl, however, permits the arrangement of a suitable pump in spite of the flat design. The piston unit can be a piston unit with a piston sliding on a cylinder surface. Due to the small installation space which is available, however, a piston unit as a membrane part which is produced at least partially from a resiliently deformable material is advantageous. A peripheral edge of the membrane part is attached to a pump chamber component so that, in particular, a central region can be pressed toward the opposing rear face by the action of the actuation handle. The pump device preferably has an inlet valve and an outlet valve, in particular in the manner of valves opening as a function of pressure. The corresponding channels, namely the inlet channel and the outlet channel, are preferably provided on the inside of the rear face of the housing.

The piston unit preferably has a resiliently deformable compensation portion, the connection being formed thereby with the actuation handle so that the pivoting movement of the actuation handle can be transferred into a translational movement of the piston.

The actuation handle itself is movable either in a purely translational manner or in a pivotably movable manner relative to the housing. Preferably, the actuation handle is highlighted in terms of color relative to the housing, in particular a surrounding housing shell.

The actuation handle can be formed by a single component which is connected via a multi-part hinge or a film hinge to a housing part. An embodiment with a multi-part actuation handle is also possible. An outer part can be provided which can be designed, in particular, from an aesthetic perspective, as well as an inner part which can serve in particular for coupling to a piston unit and is designed primarily from a technical perspective. If the actuation handle is designed in multiple parts, in particular, the outer part can be connected integrally to a housing part and/or the inner part can be connected to the housing by a multi-part hinge.

Various aspects are regarded as particularly preferred regarding the housing itself.

Thus in a preferred embodiment it is provided that the pump device has a pump chamber which is defined at least in some portions by a pump chamber component. This pump chamber component, which preferably at least partially defines an inlet from the liquid reservoir and a channel between the pump chamber and the discharge opening, is configured integrally with a reservoir wall component which forms at least substantially a defining wall of the liquid reservoir. This common integral component thus represents the main component of the dispenser and ensures, in particular, a complete seal between the wall and the pump chamber component. Moreover, such an integral design facilitates the ideal use of the interior of the housing as a liquid reservoir.

This aspect of the integral pump chamber and reservoir component can also be advantageous without the described flat design, wherein the further sub-aspects which are described in this description of the dispenser can also be additionally provided in such a dispenser without a flat design.

Preferably, in particular in an embodiment with an integral pump chamber and reservoir component of the described type, the drop dispenser has a housing shell which is separate therefrom and which is pushed onto the pump chamber component, in particular from the side of the discharge opening. The housing shell can have, in particular, a through-passage for the discharge opening, wherein it is preferred if the discharge opening is not formed directly by the housing shell but by the pump chamber and reservoir component protruding through the through-passage, or a further separate housing component. As a result, an embodiment can be achieved in which the separate housing shell does not come into contact with the liquid, as intended, so that, for example, in this regard no conditions have to be met in terms of choice of material.

If the actuation handle is configured integrally with a housing part, in particular this can be the aforementioned housing shell to which the actuation handle is pivotably articulated by means of a film hinge. Alternatively, the actuation handle can be designed in a pivotable manner by means of a multi-part hinge. In such a hinge, a hinge portion is provided on the housing side on an outer face of the pump chamber component, in particular below the described housing shell.

An intermediate space between an outer face of the pump chamber component and an inner face of the housing shell can undertake additional functions. In particular, a restoring spring can be arranged herein for restoring the actuation handle, in particular in a position which is offset relative to the pump chamber. The intermediate space can also be used in order to provide a ventilation channel here. In particular, this can be a channel which is defined at least in a partial portion over the periphery by the pump chamber component and the housing shell. Thus a design can be advantageous in which a groove is provided on an outer face of the pump chamber component, said groove being closed by the inner face of the housing shell to form a ventilation channel, in particular a capillary channel, so that a channel is formed.

One possible embodiment provides that the pump device has a pump chamber which is defined at least in some portions by a pump chamber component and that the liquid reservoir is defined by a wall which is formed at least substantially by a reservoir wall component, preferably by one of two reservoir chamber components. Here the pump chamber component and the at least one reservoir wall component are formed by at least two separate components, namely by three separate components in the case of two reservoir wall components. This makes it possible, in particular, to use a pump chamber component which is used in the same manner in different dispensers, while the reservoir wall component or the reservoir wall components is or are specifically adapted to the desired flat shape of the dispenser.

In particular, the pump chamber component can be formed by a component having a rotationally symmetrical outer contour at least in some portions, and in particular in the region of the pump chamber relative to the cross section, so that free spaces which oppose one another on both sides remain between an inner face of the flat housing and the outer contour of the pump chamber component.

Such a pump chamber component with a rotationally symmetrical outer contour in cross section can thus be used even in conventional dispensers with an approximately rotationally symmetrical outer housing. If it is used in a flat dispenser according to the invention, the aforementioned free spaces are produced.

The pump chamber component and at least the one reservoir wall component, or a reservoir wall component, preferably have interlocking contours, in particular positively interlocking contours, in the region of a liquid connection. The two components are thus directly connected to one another, wherein preferably it is a peripheral liquid-tight connection so that, as a result, the liquid path is provided from the reservoir into the pump chamber.

If an integral pump chamber and reservoir component is provided, however, it is also possible to provide a separate reservoir bottom which is tightly connected to the reservoir wall component. The reservoir bottom which is elongated due to the shape of the housing terminates the liquid reservoir on the bottom side. The reservoir bottom can represent a closure which is fastened to the reservoir component after the liquid reservoir has been filled. Preferably, however, the reservoir bottom is fixedly connected to the reservoir component, in particular by welding or adhesive bonding, even before the filling procedure. The reservoir bottom can be used for a ventilation channel instead of the pump chamber component.

In addition to the design with an integral pump chamber and reservoir component, a design can also be advantageous in which the reservoir wall component and the pump chamber component are configured as separate components and are connected to one another over the periphery, once again preferably by means of a adhesively bonded connection or a welded connection. A separate reservoir bottom component is not required in such an embodiment. The bottom can thus be formed integrally with the surrounding wall of the reservoir wall component.

It is advantageous if the reservoir wall component has a filling opening on the bottom opposing the pump chamber component. This preferably round filling opening remains open during the manufacture of the dispenser and is closed only after the liquid reservoir has been filled, preferably by means of a closure element which is snapped onto the edge of the filling opening.

The reservoir wall component, which at least partially surrounds the liquid reservoir, is preferably produced from an at least partially transparent plastics material. In particular, the reservoir wall component can have uniform material properties, so that all of the walls formed thereby are light-permeable. Both transparent and opaque embodiments and a white or different coloration are possible. The transparency makes it possible to identify easily the filling level in the liquid reservoir. The above-described housing shell can enable a light-impermeable embodiment of the housing above the liquid reservoir.

In particular polypropylene (PP), cyclic olefin copolymer (COC), cyclic olefin polymer (COP) or polyethylene terephthalate (PET) are suitable as materials for the reservoir component and the integral pump chamber and reservoir component. Preferably, virtually all other components of the drop dispenser, and in particular all of the housing parts, are produced from these or other plastics. If the springs are also produced from plastics, all of the parts can be plastics components. Preferably, the plastic parts are made of plastics which can be processed in a common recycling process.

A dispenser according to the invention preferably has an outlet valve which is arranged upstream of the discharge opening, in addition to the valves of the pump device. This prevents the ingress of germs. Preferably, the outlet valve is pressed in its closing direction by means of a closing spring and and is opened when the liquid pressure is sufficiently high. Preferably, a relatively large pressure-effective valve surface of, in particular, at least 50 mm², is provided in order to be able to use a relatively strong closing spring and yet to be able to open the valve for the liquid discharge when the liquid pressure is low. Preferably, the outlet valve is configured to open at a liquid pressure of less than 1000 mbar, in particular preferably at a pressure of less than 800 mbar or a pressure of less than 600 mbar.

### BRIEF DESCRIPTION OF THE DRAWINGS

Further advantages and aspects of the invention are found in the claims and in the following description of preferred exemplary embodiments of the invention which are described hereinafter with reference to the figures.

FIG. 1 shows a first exemplary embodiment of a drop dispenser according to the invention in an overall view.

FIGS. 2A and 2B show the drop dispenser of FIG. 1 in two sectional views.

FIG. 3 illustrates the construction of the drop dispenser of FIG. 1 consisting of individual housing parts.

FIGS. 4A and 4B show an integral pump chamber and reservoir wall component in a view which is not in section and a view which is in section.

FIGS. 5A and 5B show a second exemplary embodiment of a drop dispenser according to the invention in two sectional views.

FIGS. 6 and 7 show a third exemplary embodiment of a drop dispenser according to the invention with the protective cap removed and in position.

FIGS. 8 and 9 show a fourth exemplary embodiment of a drop dispenser according to the invention.

### DETAILED DESCRIPTION OF THE EXEMPLARY EMBODIMENTS

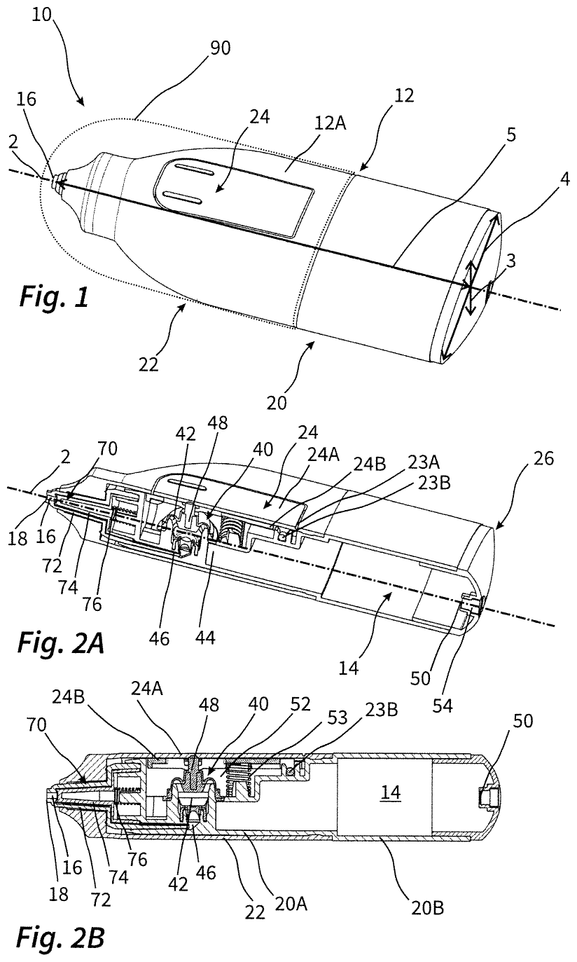

FIGS. 1 to 4B show a first embodiment of a drop dispenser according to the invention.

In FIG. 1 the drop dispenser 10 can be seen in an overall view, wherein a protective cap 90 is indicated by dotted lines.

The drop dispenser 10 has a housing 12. This housing has a length 5 in the direction of a main axis 2 which is approximately 8 cm. A width 4 of the housing 12 is approximately 4 cm. The thickness 3 of the housing is 1.8 cm. The housing 12 surrounds a liquid reservoir 14 and a pump device 40 in a manner which is not identifiable in FIG. 1.

A pivotably movable actuation handle 24 is provided on a front face 12A of the housing 12 for actuating the pump device 40. Liquid can be pumped from the liquid reservoir 14 to a discharge opening 16 by pressing in this actuation handle 24 in an overhead position. This discharge opening is surrounded by a drop-forming geometry in the form of a drop-forming ring, so that liquid which is discharged here can collect until the quantity of liquid is sufficient that a drop of the drop dispenser 10 is released from the drop-forming geometry 18.

The housing 12 has an elliptical cross section over the longest part of its length 5. Both the front face 12A and the opposing rear face are thus configured to be gently bulged toward the side. The housing 12 thus sits comfortably in the hand of the user, in particular on the ring finger, middle finger and index finger, while the actuation handle can be actuated by the thumb in a simple and reliable manner and with accurate metering.

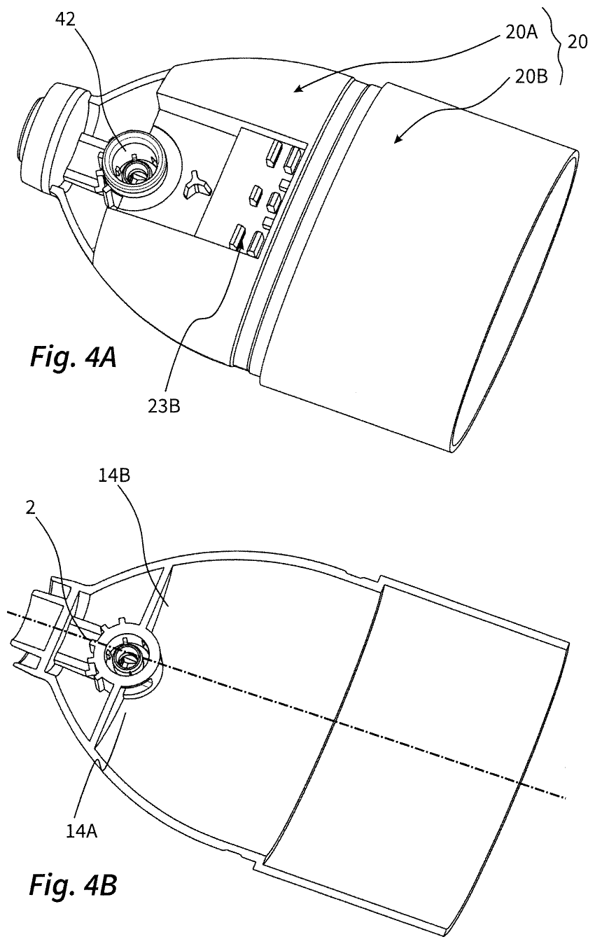

FIGS. 2A and 2B show the drop dispenser 10 in sectional view. It can be seen therefrom that the liquid reservoir 14 takes up a large part of the interior of the housing 12. As can be seen in FIG. 4B, the liquid reservoir extends in the direction of the main axis 2 toward the discharge opening 16 such that it is configured to be partially overlapping with the pump device 40. Two partial portions 14A, 14B of the liquid reservoir extend as far as the height of the pump chamber 42 of the pump device 40.

It can also be clearly identified by way of FIGS. 2A and 2B that the pump device 40 has a piston unit 48 which can be displaced orthogonally to the front face 12A and which is configured as a membrane piston unit. This means that the piston unit 48 is partially produced from a resiliently deformable material and is fastened in the edge region to the housing 12, in the present case to a pump chamber component 20A, explained below in more detail. The piston unit 48 defines the aforementioned pump chamber 42 together with the pump chamber component 20A. Pump valves are provided in the region of an inlet 44 and an outlet 46 on the input side and output side of the pump chamber 42.

If the actuation handle 24 is pressed in counter to the force of a restoring spring 53, relative to FIG. 2B the piston unit 48 is pressed downwardly thereby and liquid is pressed thereby in the direction of the discharge opening 16.

An outlet valve 70 is arranged upstream of the discharge opening 16. The primary function of this valve is to avoid the ingress of germs. The outlet valve 70 has an outlet valve nozzle 72 which forms part of the housing 12, a resiliently deformable valve body 74 which is pressed by a relatively strong valve spring 76 in the direction of a closed position being arranged in said outlet valve nozzle. The valve body 74 has a pressurizing collar which has a surface area of more than 100 mm² and thus when the liquid pressure is applied is able to open the valve counter to the force of the valve spring 76. The pressure for opening the outlet valve 70 is preferably less than 1000 mbar, in particular preferably less than 600 mbar.

The basic construction of the housing 12 of this first exemplary embodiment can be identified by way of FIGS. 2A and 2B. In the exemplary embodiment of FIGS. 1 to 4A, the housing 12 has a common pump chamber and reservoir wall component 20. This component firstly comprises the pump chamber component 20A which at least partially defines the pump chamber, and secondly is that component against which the restoring spring 53 of the actuation handle 24 and the valve spring 76 of the outlet valve 70 are supported. The reservoir wall component 20B which is configured integrally with the pump chamber component 20A forms, in particular, a cylindrical wall ring which substantially defines the liquid reservoir 14. A reservoir bottom component 26, which is inserted into the reservoir wall component 20B and is preferably welded or adhesively bonded therein, is provided on the bottom side. In the present exemplary embodiment, a ventilation channel 50 is provided in the reservoir bottom component 26 in order to introduce compensating air into the liquid reservoir after the discharge of liquid from the liquid reservoir 14. Since the liquid discharged by a drop dispenser 10 of the described type is preferably free of preservatives, the ventilation channel 50 is configured with a sterile filter 54.

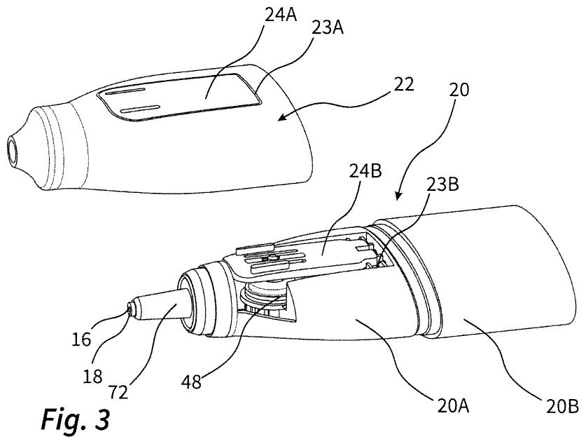

The main components of the housing 12 are described once again by way of FIG. 3. The housing 12 has the already-described pump chamber and reservoir wall component 20 with its partial portions 20A, 20B. The outlet valve nozzle 72 of the housing 12 is stationary relative to this component and is connected fixedly to the pump chamber component 20A. A recess, in which the piston unit 48 and an inner part 24B of the actuation handle 24 are arranged, is provided on the outer face of the pump chamber component 20A.

In addition, the housing 12 has the housing shell 22 on which an outer part 24A of the actuation handle 24 is provided integrally via a film hinge 23A. At opposing ends, the housing shell 22 has a through-passage for the discharge opening or the outlet valve nozzle 72 or an opening through which the pump chamber and reservoir wall component 20 can be pressed into the housing shell 22. Preferably, the components 20, 22 are latched together in the joined state.

In the joined state, the outer face of the pump chamber component 20A and the inner face of the housing shell 22 define a protected intermediate space 52 in which the restoring spring 53 is arranged and the piston unit 48 is fastened over the periphery.

FIGS. 4A and 4B show the pump chamber and reservoir wall component 20 in separate views. In FIG. 4A the component is not shown in section. It is possible to identify the shaft, which is open toward the front face and which forms the pump chamber 42 after the addition of the piston unit 48, a holding pin for the restoring spring 53 and a part of the hinge 23B. The partial portions 14A, 14B which extend relative to the main axis 2 into the region of the pump chamber 42 can be seen from the sectional view of FIG. 4B.

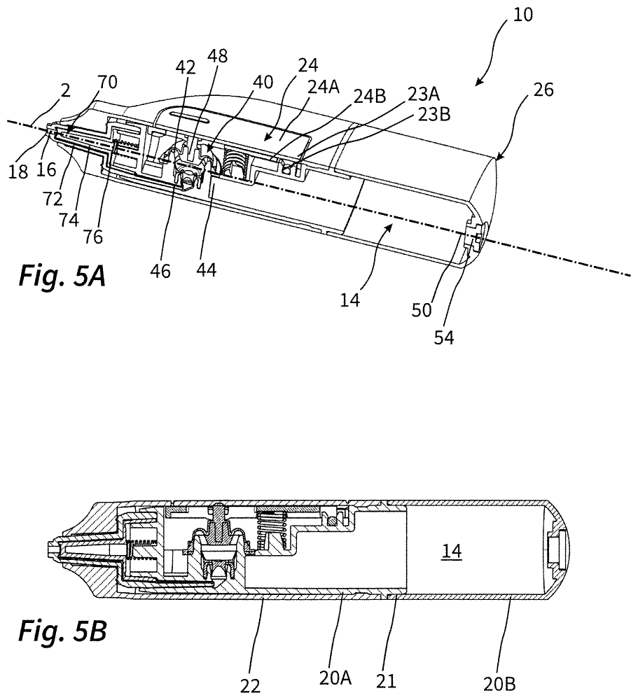

FIGS. 5A and 5B show an alternative design. Here the reservoir wall component 20B is not produced integrally with the pump chamber component 20A but the components have been connected together only retrospectively in a joining region 21, in particular by means of welding or adhesive bonding. In such an embodiment, a separate reservoir bottom component 26 can be dispensed with.

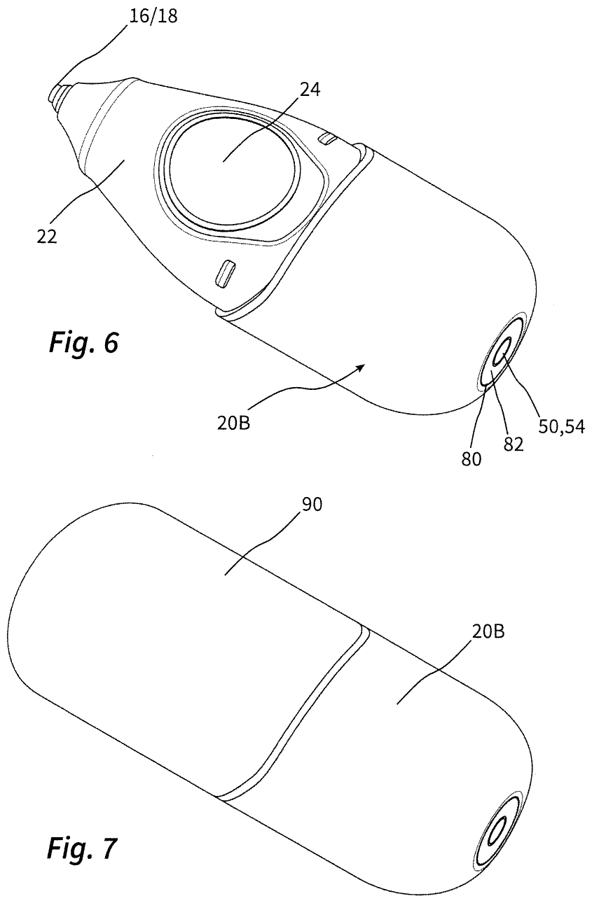

FIGS. 6 and 7 show a third exemplary embodiment of a drop dispenser 10. This drop dispenser is constructed substantially in the manner of the drop dispenser 10 of FIGS. 5A and 5B. A difference is in the general shape, in particular also the actuation handle 24. A filling opening 80, which can be closed by snapping in a closure element 82 after the filling procedure, is also provided on the bottom of the reservoir wall component 22B. The closure element 82 can comprise a ventilation channel 50 and a sterile filter 54 inserted therein.

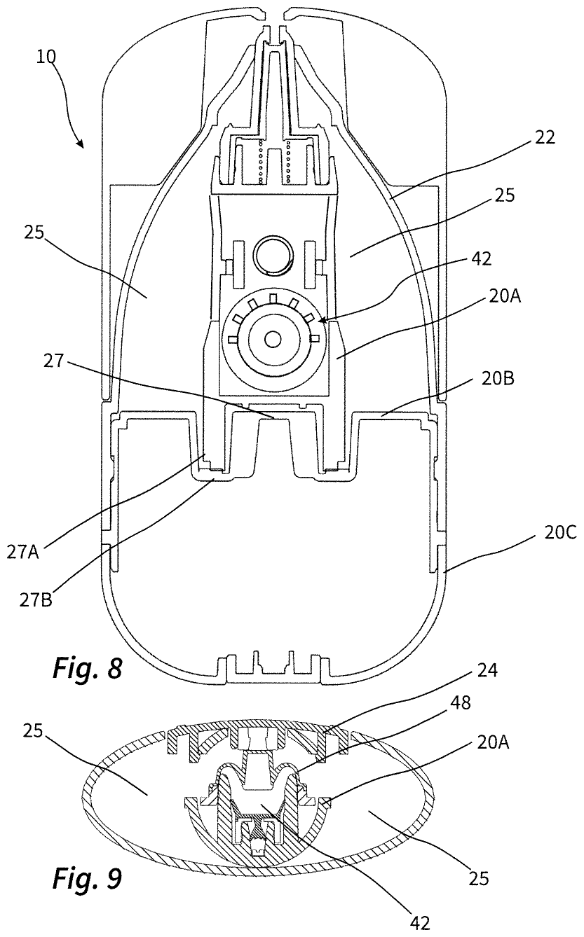

FIGS. 8 and 9 show a fourth exemplary embodiment of a drop dispenser 10 according to the invention in sectional views. The particularity of this embodiment is that separate components 20A, 20B, 20C form the pump chamber component 20A and the reservoir wall components 20B and 20C. The reservoir wall component(s) 20B and 20C are provided with a flat and preferably elliptical cross section corresponding to the flat shape of the dispenser and corresponding approximately to the embodiment of FIGS. 5A and 5B. The pump chamber component 20A which defines the pump chamber 42, however, has a substantially round outer contour as visible from FIG. 9.

As a result, it is possible to use the pump chamber component 20A with different dispensers and, in particular, also with dispensers which have an outer housing with a round cross section. This reduces the production costs since different pump chamber components 20A do not have to be provided for different dispensers.

The design results in free spaces 25 being produced between the outer contour of the pump chamber component 20A and the housing shell 22, laterally offset to the pump chamber component 20A. While these free spaces represent unused space, if this space is not used for liquid storage this results in a pleasantly low center of gravity during handling.

The reservoir chamber component 20B and the pump chamber component 20A are directly attached to one another. Preferably, these components are connected in the region of a liquid connection 27 which is located below the cutting plane of FIG. 8, in particular preferably by interlocking contours 27A, 27B. Preferably, these contours can bring about a positive coupling and thereby counteract a separation of the components 20A, 20B.

1. A drop dispenser for discharging a liquid in drops, comprising:
a liquid reservoir for storing the liquid prior to a discharge process;
a discharge opening;
a drop-forming geometry disposed adjacent the discharge opening;
a pump device for pumping liquid out of the liquid reservoir and to the discharge opening such that the liquid collects on the drop-forming geometry and is released from the drop-forming geometry when a sufficient quantity of liquid is collected;
a flat housing, the liquid reservoir and the pump device being arranged with in housing, wherein the housing has an outer shape, the outer shape of the housing having a maximum width oriented orthogonally to a main axis of the housing, said maximum width being at least 25% greater than a maximum thickness of the outer shape of the housing oriented orthogonally to the main axis and orthogonally to the maximum width; and
an actuation handle, provided on a lateral surface of the housing for actuating the pump device.

2. The drop dispenser as claimed in claim 1, wherein
the liquid reservoir extends overlappingly in a direction of the main axis into a region of the pump device.

3. The drop dispenser as claimed in claim 1,
wherein an internal volume of the liquid reservoir is at least 50%, of a volume enclosed by an outer contour of the drop dispenser.

4. The drop dispenser as claimed in claim 1, wherein
the pump device has a pump chamber component and a pump chamber defined at least in some portions by the pump chamber component,
the liquid reservoir is defined by a wall formed at least substantially by a reservoir wall component, and
the drop dispenser comprises a common component integrally forming the pump chamber component and the reservoir wall component.

5. The drop dispenser as claimed in claim 4, wherein
the drop dispenser has a housing shell pushed onto the pump chamber component, and
the housing shell has a through-passage for the discharge opening.

6. The drop dispenser as claimed in claim 1, wherein
the pump device has a pump chamber component and a pump chamber defined at least in some portions by the pump chamber component,
the liquid reservoir is defined by a wall formed at least substantially by a reservoir wall component, and
the pump chamber component and the reservoir wall component are formed by at least two separate components.

7. The drop dispenser as claimed in claim 6, wherein
the pump chamber component is formed by a component having a rotationally symmetrical outer contour at least in some portions, such that free spaces which oppose one another on both sides remain between an inner face of the housing and the outer contour of the pump chamber component.

8. The drop dispenser as claimed in claim 7, wherein
the pump chamber component and the reservoir wall component have interlocking contours in the region of a liquid connection.

9. The drop dispenser as claimed in claim 1, wherein
the drop dispenser has a reservoir wall component surrounding the liquid reservoir over a periphery, and
the drop dispenser has a reservoir bottom tightly connected to the reservoir wall component.

10. The drop dispenser as claimed in claim 1, wherein the drop dispenser has a reservoir wall component and a pump chamber component configured as separate components and connected to one another over a periphery.

11. The drop dispenser as claimed in claim 1, wherein
the drop dispenser has a reservoir wall component at least partially surrounding the liquid reservoir, and
the reservoir wall component comprises an at least partially transparent material.

12. The drop dispenser as claimed in claim 1, wherein
the housing of the drop dispenser has a maximum width of at least 2.5 cm and a maximum thickness of at most 2 cm.

13. The drop dispenser as claimed in claim 1, wherein
the pump device has a piston unit movable orthogonally to the main axis.

14. The drop dispenser as claimed in claim 1, further comprising one or more of the following:
the actuation handle comprises an outer part and an inner part; and/or
the actuation handle is articulated in a pivotably movable manner to the housing; and/or
the actuation handle or the outer part thereof is integrally connected via a hinge to a housing shell of the housing; and/or
the actuation handle or the inner part thereof is pivotably articulated to the housing by a multi-part hinge; and/or
the actuation handle or at least an outer part of the actuation handle has a coloration different from surrounding walls of the housing; and/or
the actuation handle has a width of at least 20 mm, and/or the actuation handle has a width at least 50% of the width of the housing at a widest point thereof.

15. The drop dispenser as claimed in claim 1, wherein
a cross section of the housing is provided at least in a partial region with a substantially elliptical cross section.

16. The drop dispenser as claimed in claim 1, wherein:
the housing comprises a rigid plastics material; and/or
the liquid reservoir is connected via a ventilation channel to a surrounding atmosphere, the ventilation channel comprising a sterile filter and/or a ventilation valve; and/or
the actuation handle is provided on a front face of the housing which spans the width of the housing;
the drop dispenser has a removable protective cap, the protective cap covering the actuation handle in a positioned state; and/or
the drop dispenser has an outlet valve, the outlet valve being configured to open at a liquid pressure of less than 1000 mbar; and/or
the liquid reservoir has a filling opening, the filling opening having a round shape and being closable by a closure element; and/or
the liquid reservoir is filled with a pharmaceutical liquid.

17. A drop dispenser for discharging a liquid in drops, comprising:
a liquid reservoir for storing the liquid prior to a discharge process;
a discharge opening;
a drop-forming geometry disposed adjacent discharge opening; and
a pump device for pumping liquid out of the liquid reservoir and to the discharge opening such that the liquid collects on the drop-forming geometry and is released from the drop-forming geometry when a sufficient quantity of liquid is collected;
wherein the liquid reservoir has an internal volume of at least 50%, of a volume enclosed by an outer contour of the drop dispenser.

18. a drop dispenser for discharging a liquid in drops, comprising:
a liquid reservoir for storing the liquid prior to a discharge process;
a discharge opening;
a drop-forming geometry disposed adjacent the discharge opening;
a pump device for pumping liquid out of the liquid reservoir and to the discharge opening so that the liquid collects on the drop-forming geometry and is released from the drop-forming geometry when a sufficient quantity of liquid is collected,
the pump device having a pump chamber component and a pump chamber defined at least in some portions by the pump chamber component;
the liquid reservoir is defined by a wall formed at least substantially by a reservoir wall component;
the drop dispenser further comprises a common component, the common component integrally forming the pump chamber component and the reservoir wall component.

19. The drop dispenser as claimed in claim 2, wherein the region of the pump device is a pump chamber, and the liquid reservoir has an internal volume and partial portions forming respective parts of the internal volume, one partial portion being disposed on a left-hand side of the pump chamber of the pump device and the other partial portion being disposed on a right-hand side of the pump chamber, relative to the maximum width of the housing.

20. The drop dispenser as claimed in claim 5, wherein:
the actuation handle is an integral part of the housing shell; and/or
the actuation handle is pivotably mounted on the housing shell or on an outer face of the pump chamber component; and/or
an outer face of the pump chamber component and an inner face of the housing shell define an intermediate space, the drop dispenser including a restoring spring arranged in the intermediate space for restoring the actuation handle; and/or
the drop dispenser has a ventilation channel formed at least in some portions by the pump chamber component and the housing shell, the ventilation channel being defined at least in a partial portion over a periphery by the pump chamber component and the housing shell.

21. The drop dispenser as claimed in claim 9, wherein:
the reservoir bottom is connected by an adhesively bonded connection or a welded connection to the reservoir wall component; and/or
the reservoir bottom is penetrated by a ventilation channel.

22. The drop dispenser as claimed in claim 10, wherein the pump chamber component is connected to the reservoir wall component by an adhesively bonded connection or a welded connection, and/or the reservoir wall component has on a bottom opposing the pump chamber component a filling opening closable by a closure element.

23. The drop dispenser as claimed in claim 13, wherein:
the piston unit includes a membrane part comprising a resiliently deformable material and fastened with a peripheral edge to a pump chamber component; and/or
the piston unit has a resiliently deformable compensation portion, a connection being formed thereby with the actuation handle; and/or
the pump device has an inlet from the liquid reservoir, the inlet having a narrowest side with a cross section of at least 2 mm².

24. The drop dispenser as claimed in claim 15, wherein the cross-section of the housing is provided at least over a partial region of at least 40% of a length of the housing with a substantially uniform elliptical cross-section, the cross-section of the housing tapering from this partial region to the discharge opening.

25. The drop dispenser as claimed in claim 17, wherein the internal volume of the liquid reservoir is at least 60% of the volume enclosed by the outer contour of the drop dispenser.
